(19) United States
(12) Patent Application Publication
(10) Pub. No.: US 2005/0019355 A1
(43) Pub. Date: Jan. 27, 2005

(54) Skin antiseptic and disinfectant

(76) Inventor: Robert Michael Denton, Delray Beach, FL (US)

Correspondence Address: STOCKWELL & ASSOCIATES, PSC, 861 CORPORATE DRIVE, SUITE 201, LEXINGTON, KY, 40503, US

(21) Appl. No.: 10/625,765

(22) Filed: Jul. 23, 2003

(51) Int. Cl. A61K 31/22; A61K 7/00

(52) U.S. Cl. 424/401; 514/546

(57) ABSTRACT

A skin antiseptic and disinfectant composition and its method of use are disclosed. The inventive antiseptic and disinfectant comprises an effective concentration of at least one alkyl ester of lactic acid, which is suitable for reducing bacterial count on the surface of the skin. Disinfection of the skin is achieved by applying topically the compositions to the surface of the skin to reduce the bacterial count on the skin.

### BACKGROUND OF THE INVENTION

This invention relates generally to the field skin antiseptic and disinfectant compositions and more specifically to compositions comprising alkyl lactates and the process for using same to reduce the bacterial count of the skin.

Various antimicrobials are known for use in antiseptic and disinfectant formulations including alcohols, the quaternary ammonium compounds (e.g. Benzalkonium chloride), iodine formulations, phenolic compounds (e.g. triclosan and hexachlorophene), and bisbiguanides (e.g. chlorhexidene gluconate). Such antimicrobial ingredients are also well-known additives for a variety of products, such as deodorant soap bars, underarm deodorants and liquid soaps.

In accordance with the present invention, it has been discovered that compositions based on alkyl lactate esters have antibacterial properties. Alkyl lactates are non-toxic and biodegradable. In fact, Alkyl lactates are widely used as food additives, in pharmaceuticals and cosmetics.

The use in topical compositions of short chain alkyl lactates to treat acne or long chain lactate esters as emollients is broadly taught in the art. However, no art teaches methods or compositions for utilizing the surprising antibacterial action of the alkyl lactates.

U.S. Pat. Nos. 6,322,799 and 5,961,992 teaches compositions comprising mixtures of short and long chain alpha-hydroxy esters for moisturizing the skin in the short and long term. Compositions comprising alkyl lactates for reducing the bacterial count on the skin are not taught.

U.S. Pat. No. 4,540,567 teaches compositions comprising short chain alkyl lactates for treatment of acne. There is no teaching or suggestion for compositions or methods utilizing alkyl lactates to reduce the bacterial count of skin.

U.S. Pat. No. 5,002,680 teaches skin cleansing aerosol mousse compositions with moisturization benefits. Lactic acid esters are among moisturizers taught which can be used in formulations comprising surfactant, polymeric skin feel aid and propellant. However, there is no teaching or suggesting the utilization of alkyl lactates to reduce the bacterial count of skin.

U.S. Pat. No. 4,529,605 teaches a bathing oil composition comprising a nonionic surfactant, a cationic surfactant and a primary emollient which may be a long chain lactate ester. There is no teaching or suggesting the utilization of alkyl lactates to reduce the bacterial count of skin.

U.S. Pat. No. 4,234,599 to Van Scott et al. relates to a method of treating dry skin by topically applying a composition containing esters of hydroxy acids. There is no teaching or suggesting the utilization of alkyl lactates to reduce the bacterial count of skin.

The present invention addresses several of the limitations found in the skin antiseptic and disinfectants of the prior art. Topical exposure to quaternary ammonium compounds, phenolic compounds and bisbiguanide may trigger hypersensitivity reactions, such as contact dermatitis, in certain individuals. These sensitive individuals must avoid future contact with the triggering compound. Therefore, the invention of alternative antibacterial compositions is desirable.

Alcohols are most effective as antibacterials at concentrations between 60 to 90 percent. However, the use of high concentrations of alcohols causes chapping and cracking of the skin of the user. Generally, a concentration of less than 70 percent must he used to minimize skin irritation. Alcohol compositions may be combined with moisturizers to ameliorate these undesirable effects; however, the narrow range of effective concentrations of alcohols drastically limits the possible combinations. Therefore, it is desirable to invent an antibacterial composition that is more amenable to being combined with other ingredients.

Iodine preparations may cause irritation and staining of the skin.

Certain phenolic antiseptic compositions raise serious environmental and health concerns. Due principally to the synthesis chemistry of polychloro-diphenyl ethers and phenoxy phenols, there is the potential for the formation of small amounts of unwanted trace by-products. Hexachlorophene and certain polychloro-phenoxy phenols have been observed to contain various low levels of polychlorinated dioxins and polychlorinated furans. Since triclosan is a polychloro phenoxy phenol, it may also be subject to contamination with dioxins and dibenzofurans in varying low level amounts as synthesis impurities. If present, their relative concentrations as impurities can vary from batch to batch. This raises concerns because of the toxicity of dioxins and dihenzofurans.

Hexachlorophene itself can he toxic and, in some cases, fatal when applied repeatedly to the skin, particularly in infants. Confusion, lethargy and convulsions may occur. Tetratogenic effects have been reported in pregnant women routinely using hexachlorophene. Hexachlorophene is used by health care personnel in washing and in preparing the skin of surgical patients. Because of potential neurotoxicity, it is no longer used with infants.

Accordingly, it is a primary object of the invention to provide skin antiseptic and disinfectant compositions that are relatively nontoxic. Another object of the invention is to provide skin antiseptic and disinfectant compositions that are biodegradable. Another object of the invention is to provide antibacterial compositions that are readily combined with other ingredients. Another object of the invention is to provide an antibacterial composition that does not stain the skin.

Other objects and advantages of the present invention will become apparent from the following descriptions wherein, by way of illustration and example, an embodiment of the present invention is disclosed.

### BRIEF SUMMARY OF THE INVENTION

In accordance with a preferred embodiment of the invention, there is disclosed a skin antiseptic and disinfectant compositions comprising alkyl esters of lactic acid.

Also, in accordance with a preferred embodiment of the invention, there is disclosed a method reducing the bacterial count of the skin comprising applying compositions comprising alkyl lactate to the surface of the skin.

### DETAILED DESCRIPTION OF THE INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system structure or manner.

In accordance with the present invention, it has been discovered that compositions based on alkyl lactate esters have remarkable antibacterial properties. The compositions of the invention comprise alkyl lactates wherein the alkyl group has 1 to 12 carbon atoms.

According to one preferred embodiment of the present invention, the antibacterial composition contains ethyl lactate as the antimicrobial ingredient. Antimicrobial activity of the composition is attained at concentrations of ethyl lactate greater than two per cent, achieving maximal antibacterial activity at ethyl lactate concentrations of approximately fifty percent. Accordingly, the preferred concentration range for ethyl lactate when employed in the antimicrobial compositions is between two and fifty percent. The composition of the invention may contain one or several other compounds with antiseptic properties, perfumes or other customary additives and auxiliaries such as emollients, water, odorants, surfactants, disinfectants, detergents or a combination thereof. The compositions according to the invention can be prepared by mixing the individual components together successively, if necessary with heating. No particular order need be adhered to during this process.

### EXAMPLE 1

The following example demonstrates that ethyl lactate is a potent bactericide. Using a modification of AOAC Method 956.17, the lower effective concentration for ethyl lactate was tested against E. coli (ATCC 25922) at three reaction times at varying concentrations of ethyl lactate. As the results in Table 1 show, exposure of E. coli to a 15% solution of ethyl lactate for 10 minutes eliminated bacterial growth.

TABLE 1
Lower effective concentration of ethyl lactate against
E. Coli (ATCC 25922) at final concentration of
CA. 100 billion colonies for 24 hours.

| | |
|---|---|
| Ethyl lactate, | Time |

| Label | concentration^{a} | 5 min. | 10 min. | 15 min. |
|---|---|---|---|---|
| S-1^{b} | 10% | +^{c} | + | + |
| S-2 | 50% | −^{d} | − | − |
| S-3 | 20% | − | − | − |
| S-4 | 30% | − | − | − |
| S-5 | 40% | − | − | − |
| S-6 | 15% | + | − | − |
| S-7 | 20% | − | − | − |

^{a}Diluted with water, plus challenge bacteria (0.5 ml of 1 trillion E. Coli added to 5.0 ml of each sample).
^{b}S = Sample
^{c}+ = Bacterial growth observed
^{d}− = No Bacterial growth observed

### EXAMPLE 2

The following example shows one preferred embodiment according to the invention. A formulation is prepared as follows:

| Ingredient | Weight % |
|---|---|
| Ethyl Lactate | 16.0% |
| Fragrance | 1.0% |
| Water | 83.0% |

Fragrance is added first to the ethyl lactate and then mixed in the water.

Turning to Table 2 there is shown that the bactericidal efficiency of the formulation in Example 2 is comparable to that of 10% Clorox at the challenge times observed.

TABLE 2
Comparison of preferred embodiment with diluted Clorox
against E. coli (ATCC 25922) at concentration of
CA. 100 billion colonies for 24 hours.

| | | Time | | |
|---|---|---|---|---|
| Label | concentration^{a} | 5 min. | 10 min. | 15 min. |
| Clorox | 10% | N^{b} | N | N |
| Preferred embodiment | 100% | N | N | N |

^{a}Diluted with water, plus challenge bacteria (0.5 ml of 1 trillion E. Coli added to 5.0 ml of each sample).
^{b}N = No Bacterial growth observed

Turning to Table 3 there is shown with a conventional challenge test that the formulation eliminates E. Coli with a very high efficiency.

TABLE 3
Results of challenge test using E. coli (ATCC 25922).

| | | |
|---|---|---|
| Challenge volume: | 5.0 | ml |
| Product volume: | 0.5 | ml |
| E. Coli level of challenge liquid: | 100 | Billion CFU/ml* |
| E. Coli level of treated liquid: | <1 | CFU/ml |

| | |
|---|---|
| E. Coli removal efficiency: | >99.999% |

*CFU/ml = Colony forming unit per milliliter.

It is thus apparent from the results of the described Examples that the antibacterial according to the invention is a valuable chemical disinfectant possessing good bactericidal effects. The skin sanitizer according to the present invention is particularly well-suited also in medical and food preparation contexts.

It is well known in the art that compositions comprising alkyl lactate are safe for topical use. One skilled in the art will appreciate that the particular alkyl lactate chosen can provide a more immediate and more effective reduction in bacterial count depending on the bacterial microflora targeted. Accordingly, the described examples are merely exemplary and are in no way limiting.

The concentration of alkyl lactate in accordance with the present invention should be sufficient to effect the desired reduction in bacterial count over a reasonable time frame. One skilled in the art will recognize that concentration will depend upon a variety of factors, including the particular alkyl lactate employed, the targeted bacterial microflora, and the nature of the other compounds in the bacterial microflora may require prolonged treatment involving multiple applications of compositions of the present invention.

Suitable concentrations of alkyl lactate can be determined by conventional range-finding techniques known to those of ordinary skill in the art. Several standard methods for determining the bactericidal efficacy of various concentrations of alkyl lactates as applied to the skin on the various resident and transient bacterial microflora thereon are well known to those of ordinary skill in the art. By way of example Table 4 presents a list of such methods, by title, propounded by the American Society for Testing and Materials (ASTM), the contents of which are herein incorporated by reference.

TABLE 4
ASTM Standard Test Methods

```
1. Standard Test Method for Assessment of an Antibacterial
  Handwash Product by Multiple Basin Wash Technique.
2. Standard Test Method for Evaluation of Antibacterial
  Washes by Cup Scrub Technique
3. Standard Test Method for Evaluation of Health Care
  Personnel Handwash Formulation
4. Standard Test Method for Evaluation of a Pre-Operative
  Skin Preparation
5. Standard Test Method for Evaluation of Surgical Hand
  Scrub Formulations
6. Standard Test Method for the Assessment of an
  Antibacterial Handwash Product by Multiple Basin Wash
  Technique
7. Evaluation of Antibacterial Washes by Agar Patch
  Technique
```

Generally, testing is initiated with smaller concentrations of alkyl lactate, which are less than the optimum concentration of the compound. Thereafter, the concentration is increased by small increments until the optimum effect under the circumstances is reached. The present inventive method will typically involve the concentrations of from about 2 to about 50 percent. With optimum efficiency ranging from about 10 to about 20 percent.

Seen as a whole, therefore, the compositions according to the invention are especially suitable as skin antiseptics and hand disinfectants. The invention has been described in detail with particular reference to preferred embodiments thereof, but it will be understood that variations and modifications can be effected within the spirit and scope of the invention.

1. A composition for disinfection of the skin comprising:
at least one alkyl ester of lactic acid wherein the alkyl group of said alkyl ester has 1 to 12 carbon atoms.

2. The composition of claim 1 wherein the alkyl ester of lactic acid is methyl lactate.

3. The composition of claim 1 wherein the alkyl ester of lactic acid is butyl lactate.

4. The composition of claim 1 wherein the alkyl ester of lactic acid is propyl lactate.

5. The composition of claim 1 wherein the alkyl ester of lactic acid is ethyl lactate.

6. The composition of claim 2 wherein the concentration of methyl lactate is between about 2% and about 50% by volume.

7. The composition of claim 3 wherein the concentration of butyl lactate is between about 2% and about 50% by volume.

8. The composition of claim 4 wherein the concentration of propyl lactate is between about 2% and about 50% by volume.

9. The composition of claim 5 wherein the concentration of ethyl lactate is between about 2% and about 50% by volume.

10. The composition of claim 1 further comprising a composition selected from the group comprising emollients, water, odorants, surfactants, disinfectants, detergents or a combination thereof.

11. The composition of claim 6 further comprising:
a composition selected from the group comprising emollients, water, odorants, surfactants, disinfectants, detergents or a combination thereof.

12. The composition of claim 7 further comprising:
a composition selected from the group comprising emollients, water, odorants, surfactants, disinfectants, detergents or a combination thereof.

13. The composition of claim 8 further comprising:
a composition selected from the group comprising emollients, water, odorants, surfactants, disinfectants, detergents or a combination thereof.

14. The composition of claim 9 further comprising:
a composition selected from the group comprising emollients, water, odorants, surfactants, disinfectants, detergents or a combination thereof.

15. A composition comprising:
at least one alkyl ester of lactic acid wherein alkyl group is selected from methyl, butyl, propyl, or ethyl in concentrations between 10% and about 20% by volume.

16. The composition of claim 15 further comprising:
a composition selected from the group emollients, water, odorants, surfactants, disinfectants, detergents or a combination thereof.

17. A method for reducing the bacterial count of the skin comprising:
applying topically to the surface of the skin a composition of at least one alkyl ester of lactic acid wherein the alkyl group of said alkyl ester has 1 to 12 carbon atoms.

18. The method of claim 17 wherein the alkyl ester of lactic acid is methyl lactate.

19. The method of claim 17 wherein the alkyl ester of lactic acid is butyl lactate.

20. The method of claim 17 wherein the alkyl ester of lactic acid is propyl lactate.

21. The method of claim 17 wherein the alkyl ester of lactic acid is ethyl lactate.

22. The method of claim 18 further comprising:
applying topically to the surface of the skin a composition wherein the concentration of methyl lactate is between about 2% and about 50% by volume; and
wherein said composition is selected from the group comprising emollients, water, odorants, surfactants, disinfectants, detergents or a combination thereof.

23. The method of claim 19 comprising:
applying topically to the surface of the skin a composition wherein the concentration of butyl lactate is between about 2% and about 50% by volume; and
wherein said composition is selected from the group comprising emollients, water, odorants, surfactants, disinfectants, detergents or a combination thereof.

24. The method of claim 20 comprising:
applying topically a composition wherein the concentration of propyl lactate is between about 2% and about 50% by volume; and wherein said composition is selected from the group comprising emollients, water, odorants, surfactants, disinfectants, detergents or a combination thereof.

25. The method of claim 21 comprising:
applying topically a composition wherein the concentration of ethyl lactate is between about 2% and about 50% by volume; and wherein said composition is selected from the group comprising emollients, water, odorants, surfactants, disinfectants, detergents or a combination thereof.

26. A method for reducing the bacterial count of the skin comprising:
applying topically to the surface of the skin a composition with at least one alkyl ester of lactic acid wherein alkyl group is methyl, butyl, propyl, or ethyl in concentrations between 10% and about 20% by volume; and a composition selected from the group comprising emollients, water, odorants, surfactants, disinfectants, detergents or a combination thereof.
